(19) United States
(12) Patent Application Publication
(10) Pub. No.: US 2013/0023513 A1
(43) Pub. Date: Jan. 24, 2013

(54) Methods and Compositions for Treating Cardiovascular Disorders

(71) Applicant: Thomas E. Hughes, Concord, MA (US)

(72) Inventor: Thomas E. Hughes, Concord, MA (US)

(21) Appl. No.: 13/521,530

(22) PCT Filed: Jan. 11, 2011
(86) PCT No.: PCT/US2011/020866

§ 371 Date: Oct. 10, 2012

Related U.S. Application Data
(60) Provisional application No. 61/294,216, filed on Jan. 12, 2010.
(60) Provisional application No. 61/315,465, filed on Mar. 19, 2010.
(60) Provisional application No. 61/379,517, filed on Sep. 2, 2010.

(51) Int. Cl. A61K 31/336; A61P 9/10; A61P 9/04; A61P 25/00; A61P 3/06; A61K 31/505; A61K 31/397; A61K 31/366; A61K 31/40; A61K 31/4418; A61K 31/404; A61K 31/47; A61P 9/00; A61K 31/455

(52) U.S. Cl. 514/210.02; 514/475; 514/356; 514/460; 514/423; 514/277; 514/415; 514/311; 514/275

(57) ABSTRACT

The invention generally relates to methods of treating a patient having, and/or at risk of, cardiovascular or cerebrovascular disorders. Such methods may include administering a MetAP2 inhibitor at a dose that does not substantially modulate angiogenesis.

### REFERENCE TO RELATED APPLICATIONS

This application claims priority to U.S. Ser. No. 61/294,216, filed Jan. 12, 2010, U.S. Ser. No. 61/315,465, filed Mar. 19, 2010, and U.S. Ser. No. 61/379,517, filed Sep. 2, 2010, each of which is incorporated by reference in its entirety.

### BACKGROUND

Cardiovascular diseases, such as atherosclerotic disease, are a major cause of mortality. Risk factors include elevated plasma total or LDL cholesterol, elevated triglycerides, low HDL cholesterol, e.g. hyperlipidemia, hypercholesterolemia, or hypoalphalipoproteinemia, and increased inflammatory markers such as C-reactive protein and fibrinogen.

Obese, and to some extent, overweight patients may have an even higher risk of such cardiovascular diseases. For example, obesity itself may raise blood cholesterol and/or triglycerides. Obesity may increase the risk of developing type 2 diabetes, which itself may increase risk of cardiovascular disease. Type 2 diabetes is a metabolic disorder that is primarily characterized by insulin resistance, relative insulin deficiency, inflammation, and hyperglycemia.

Subjects suffering from type 2 diabetes are also likely to have dyslipidemia (i.e., diabetic dyslipidemia), where the subjects have abnormally low levels of HDL and/or abnormally high levels of low density lipoprotein (LDL), cholesterol, and/or abnormally high levels of triglycerides. The low levels of HDL (i.e. <40 mg/dL) and/or high levels of LDL (i.e. >100 mg/dL) and/or high levels of triglycerides (i.e. >150 mg/dL) increase the risk of atherosclerosis and the risk for developing cardiovascular disease.

Unfortunately, standard treatments for dyslipidemia, such as niacin or fibric acid, can be contradicted in e.g., diabetic patients. Niacin is a water-soluble vitamin whose derivatives play essential roles in energy metabolism in the living cell and in DNA repair, and when taken in large doses, blocks the breakdown of fats in adipose tissue, thus altering blood lipid levels. Niacin appears to target the GPR109A receptor (sometimes referred to as PUMA, HM74, niacin receptor 1 or NIACR1 receptor). Activation (e.g. by niacin) of the GPR109A receptor can block the breakdown of fats therefore causing a decrease in free fatty acids in the blood and consequently, decreased secretion of VLDL and cholesterol by the liver.

High dose niacin has been shown to elevate fasting blood sugar levels, thereby worsening type 2 diabetes. Accordingly, niacin is typically contra-indicated for persons with type 2 diabetes. The presence of niacin may contribute to niacin-induced insulin resistance.

For patients, especially obese and/or diabetic patients, there is a dramatic need for new methods of treating cardiovascular disorders.

### SUMMARY

A method of treating, or minimizing the risk of, cardiovascular disease (for example atherosclerosis, heart attack, stroke, or heart failure) in a patient in need thereof, comprising administering to said patient an therapeutically effective amount of a MetAP2 inhibitor thereby increasing serum levels of high density lipoproteins in said patient, wherein said therapeutically effective amount does not substantially modulate or suppress angiogenesis. The patient being treated may be for example, obese, overweight, and/or suffering from diabetes, e.g. type 2 diabetes.

In another embodiment, a method of reducing triglycerides in the serum of a patient in need thereof (e.g. an obese and/or diabetic patient) is provided, comprising administering to said patient a therapeutically effective amount of a MetAP2 inhibitor, wherein said therapeutically effective amount does not substantially modulate or suppress angiogenesis. In a particular embodiment, a method is provided for improving, or increasing high density lipoprotein (HDL) in the serum of a patient, that includes administering to a patient a therapeutically effective amount of a MetAP2 inhibitor.

For example, contemplated patients benefiting from disclosed methods may be contradicted from administration of hypolipidemic agents such as statins, a cholesteryl ester transfer protein inhibitor, niacin and/or a GPR109a receptor agonist, and/or from amounts of these agents necessary to show improvement.

In one aspect, a method of treating, or minimizing the risk of, cardiovascular or cerebrovascular disease in a patient (e.g., a patient at risk for cardiovascular or cerebrovascular disease) in need thereof is provided, comprising administering to said patient an therapeutically effective amount of a MetAP2 inhibitor, wherein said therapeutically effective amount does not substantially modulate or suppress angiogenesis.

In another aspect, a method of decreasing the level of one or more markers indicative of cardiovascular disease risk in a patient in need thereof is provided, comprising administering to said patient a therapeutically effective amount of a MetAP2 inhibitor. Markers indicative of cardiovascular disease risk include C-Reactive Protein (CRP), low density lipoprotein cholesterol (LDL-c), lipoprotein(a)(Lp(a)), and thrombomodulin.

In another embodiment, a method of reducing Low Density Lipoprotein Cholesterol (LDL-cholesterol) in the serum of a patient in need thereof (e.g. an obese and/or diabetic patient) is provided, comprising administering to said patient a therapeutically effective amount of a MetAP2 inhibitor, wherein said therapeutically effective amount does not substantially modulate or suppress angiogenesis. In a particular embodiment, a method is provided for improving, or lowering low density lipoprotein (LDL) in the serum of a patient, that includes administering to a patient a therapeutically effective amount of a MetAP2 inhibitor.

In another embodiment, a method of reducing Lipoprotein(a) in the serum of a patient in need thereof (e.g. an obese and/or diabetic patient) is provided, comprising administering to said patient a therapeutically effective amount of a MetAP2 inhibitor, wherein said therapeutically effective amount does not substantially modulate or suppress angiogenesis. In a particular embodiment, a method is provided for improving, or lowering lipoprotein(a) (Lp(a)) in the serum of a patient, that includes administering to a patient a therapeutically effective amount of a MetAP2 inhibitor.

In another embodiment, a method of reducing C-reactive protein in the serum of a patient in need thereof (e.g. an obese and/or diabetic patient) is provided, comprising administering to said patient a therapeutically effective amount of a MetAP2 inhibitor, wherein said therapeutically effective amount does not substantially modulate or suppress angiogenesis. In a particular embodiment, a method is provided for improving, or lowering C-reactive protein (CRP) in the serum of a patient, that includes administering to a patient a therapeutically effective amount of a MetAP2 inhibitor.

Also provided herein is a method of treating a diabetic patient suffering from hypercholesterolemia, hyperlipidemia, and/or hypoalphalipoproteinemia, comprising administering to said patient a therapeutically effective amount of a MetAP2 inhibitor, and which may further, optionally comprise administering niacin, statins, cholesteryl ester transfer protein inhibitors, or other lipid modulating agents to said patient. Such therapeutically effective amount may not, in some embodiments, substantially modulate or suppress angiogenesis. For example, a method of treating hyperlipidemia and/or hypercholesterolemia in a patient in need thereof is provided that comprises administering an effective amount of the following to said patient: a) one or more therapeutic agents each selected from the group consisting of: niacin, a statin, a fibrate, an angiotension-converting enzyme inhibitor, and a cholesterol absorption inhibitor (e.g., ezetimibe, simvastatin, atorvastatin, cerivastatin, fluvastatin, lovastatin, mevastatin, pitavastatin, rosuvastatin, bezafibrate, ciprofibrate, clofibrate, gemfibrozil, anacetrapib, and fenofibrate); and b) a MetAP-2 inhibitor.

MetAP2 inhibitors may be a substantially irreversible inhibitor, e.g. a fumagillin, fumagillol or fumagillin ketone derivative, siRNA, shRNA, an antibody, or a antisense compound, or may be a substantially reversible inhibitor. For example, an MetAP2 inhibitor may be selected from O-(4-dimethylaminoethoxycinnamoyl)fumagillol and pharmaceutically acceptable salts thereof.

In another embodiment, a method of minimizing undesired side effects such as flushing associated with therapeutic administration of niacin or a GPR109a receptor agonist in a patient in need thereof is provided that includes administering to said patient a therapeutically effective amount of a MetAP-2 inhibitor. In some embodiments, a method of lowering total cholesterol levels (and/or increasing high density lipoproteins) is provided comprising administering an effective amount niacin and an effective amount of a MetAP-2 inhibitor, whereby the MetAP-2 inhibitor provides for a lower effective amount of niacin as compared to an amount that would be effective if niacin is administered alone.

### BRIEF DESCRIPTION OF FIGURES

FIG. 1A is a bar graph showing the concentration of beta-hydroxybutyrate in mice fed a high fat diet (vehicle) or mice fed a high-fat diet plus oral administration of fumagillin for ten days at the concentrations indicated. FIG. 1B is a bar graph showing the concentration of nonesterified fatty acid concentrations under the conditions described for FIG. 1A. FIG. 1C is a bar graph showing the concentration of beta-hydroxybutyrate in mice fed a high fat diet (vehicle) or mice fed a high-fat diet plus oral administration of fumagillin for 250 days at the concentrations indicated.

FIG. 2 contains three bar graphs showing concentrations of glucose (top left graph), insulin (top right graph) and glucose-insulin complex (bottom graph) in mice fed a high-fat diet (diet-induced obesity (DIO), left bar), mice fed a high-fat diet plus 1 mg/kg fumagillin (DIO+ZGN-201, middle bar), and age-matched lean mice (right bar).

FIG. 3 indicates the effect of MetAP2 inhibitor treatment on plasma total cholesterol in humans.

FIG. 4 depicts mouse FGF-21 plasma levels after treatment with fumagillin.

FIG. 5 depicts hepatic PCSK9 mRNA levels after treatment with fumagillin.

FIG. 6 depicts CRP levels after treatment with 6-O-(4-dimethylaminoethoxy)cinnamoyl fumagillol oxalate.

FIG. 7. depicts thrombomodulin levels in patients after treatment with 6-O-(4-dimethylaminoethoxy)cinnamoyl fumagillol oxalate.

FIG. 8. depicts LDL levels in patients after treatment with 6-O-(4-dimethylaminoethoxy)cinnamoyl fumagillol oxalate.

FIG. 9. depicts Apo(a) levels in patients after treatment with 6-O-(4-dimethylaminoethoxy)cinnamoyl fumagillol oxalate.

### DETAILED DESCRIPTION

### Overview

The disclosure relates at least in part to methods for treating a patient suffering from, or at risk of, cardiovascular, cerebrovascular, or heart disease. For example, provided herein are methods of treating atherosclerosis, heart attack, stroke, heart failure, hypercholesterolemia, and/or hyperlipidemia, in a patient in need thereof, which include administering an effective amount of a MetAP2 inhibitor.

MetAP2 encodes a protein that functions at least in part by enzymatically removing the amino terminal methionine residue from certain newly translated proteins. Increased expression of the MetAP2 gene has been historically associated with various forms of cancer. Molecules inhibiting the enzymatic activity of MetAP2 have been identified and have been explored for their utility in the treatment of various tumor types and infectious diseases such as microsporidiosis, leishmaniasis, and malaria. However, cardiovascular disease is typically not a form of cancer, and it has been found that MetAP2 inhibitors can effectively treat subjects with, or at risk of, cardiovascular disease. Disclosed herein are methods relating to administering a MetAP-2 inhibitor to treat heart disease, e.g., by administering an effective amount of a MetAP-2 inhibitor, e.g. a therapeutically effective amount but that does not substantially modulate or suppress angiogenesis.

Without being limited by any particular theory or mechanism of action, it is believed that fat oxidation and lipolysis are stimulated through treatment with inhibitors of MetAP2 which may over-ride the inhibitory effects of hyperinsulinemia on production of ketone bodies related at least in part to insulin-stimulation and/or over-rides the inhibitory effects of high fat diet induced NADPH oxidase activity. A coordinated action can be induced which leads to a physiological reduction in body adiposity through one or more actions including increased loss of fat tissue-associated triglyceride, with corresponding enhanced activity of brown adipose tissue and its sensitivity to physiological stimuli, increased metabolism of free fatty acids by the liver with increased ketone body formation, and reduced food intake. These effects are evident at doses of a MetAP2 inhibitor that do not substantially modulate angiogenesis.

Administration of a MetAP2 inhibitor, without being limited by any theory, suppresses activity of the extracellular regulated kinases ERK1/2, suppressing pathways controlling fatty acid synthesis, sterol synthesis and inflammation leading to increased fatty acid oxidation and elevated ketone body production. Because beta-hydroxybutyrate appears to be a ligand for the GPR109A receptor, elevated beta-hydroxybutyrate concentrations may exert a nicotinic acid (niacin) like effect; i.e., administration of MetAP2 inhibitors may e.g. lower cholesterol and/or impart a niacin like improvement in cardiovascular disease risk including elevation of circulating high density lipoprotein concentrations.

Beta-hydroxybutyrate may also assist in controlling fatty acid release, e.g. by modulating the GPR109A receptor, and therefore the administration of MetAP2 may improve HDL concentrations without significant flushing. For example, the MetAP2 mechanism may exert a ‘self-braking’ effect related to the feedback loop governed by the control of fatty acids, which serve as a substrate for beta-hydroxybutyrate synthesis. This feedback mechanism may work to limit GPR109A receptor activation within an effective range without over-stimulation receptor activity. Administration of MetAP2 inhibitors may allow the use of niacin or other GPR109A receptor modulators in patients previously contraindicated from such use, e.g. patients with type 2 diabetes, in part (without being limited by any theory) because such MetAP2 inhibitors improve glucose tolerance. Currently, administration of e.g., niacin alone is contradicted in patients with diabetes because of worsening of glucose tolerance.

As a component of its effect on genes controlling cholesterol production, the MetAP2 mechanism further can suppress production of proprotein convertase subtilisin/kexin type 9, or PCSK9, which is a negative modulator of low density lipoprotein receptor function. Reduction of PCSK9 expression and production restores LDL receptor activity, thereby lowering LDL cholesterol levels. Administration of MetAP2 inhibitors therefore may allow for more effective management of LDL cholesterol and cardiovascular risk.

### MetAP2 Inhibitors

MetAP2 inhibitors refer to a class of molecules that inhibit or modulate the activity of MetAP2, e.g., the ability of MetAP2 to cleave the N-terminal methionine residue of newly synthesized proteins to produce the active form of the protein, or the ability of MetAP2 to regulate protein synthesis by protecting the subunit of eukaryotic initiation factor-2 (eIF2) and/or ERK1/2 from phosphorylation.

Exemplary MetAP2 inhibitors may include irreversible inhibitors that covalently bind to MetAP2. For example, such irreversible inhibitors include fumagillin, fumagillol, and fumagillin ketone.

Derivatives and analogs of fumagillin, and pharmaceutically acceptable salts thereof are contemplated herein as irreversible MetAP2 inhibitors, such as O-(4-dimethylaminoethoxycinnamoyl)fumagillol (also referred to herein as Compound A), O-(3,4,5-trimethoxycinnamoyl)fumagillol, O-(4-chlorocinnamoyl)fumagillol; O-(4-aminocinnamoyl)fumagillol; O-(4-dimethylaminoethoxycinnamoyl)fumagillol; O-(4-methoxycinnamoyl)fumagillol; O-(4-dimethylaminocinnamoyl)fumagillol; O-(4-hydroxycinnamoyl)fumagillol; O-(3,4-dimethoxycinnamoyl)fumagillol; O-(3,4-methylenedioxycinnamoyl)fumagillol; O-(3,4,5-trimethoxycinnamoyl)fumagillol; O-(4-nitrocinnamoyl)fumagillol; O-(3,4-dimethoxy-6-aminocinnamoyl)fumagillol; O-(4-acetoxy-3,5-dimethoxycinnamoyl)fumagillol; O-(4-ethylaminocinnamoyl)fumagillol; O-(4-ethylaminoethoxycinnamoyl)fumagillol; O-(3-dimethylaminomethyl-4-methoxycinnamoyl)fumagillol; O-(4-trifluoromethylcinnamoyl)fumagillol; O-(3,4-dimethoxy-6-nitrocinnamoyl)fumagillol; O-(4-acetoxycinnamoyl)fumagillol; O-(4-cyanocinnamoyl)fumagillol; 4-(4-methoxycinnamoyl)oxy-2-(1,2-epoxy-1,5-dimethyl-4-hexenyl)-3-methoxy-1-chloromethyl-1-cyclohexanol; O-(3,4,5-trimethoxycinnamoyl)fumagillol; O-(4-dimethylaminocinnamoyl)fumagillol; O-(3,4,5-trimethoxycinnamoyl)oxy-2-(1,2-epoxy-1,5-dimethyl-4-hexenyl)-3-m-ethoxy-1-chloromethyl-1-cyclohexanol; O-(4-dimethylaminocinnamoyl)oxy-2-(1,2-epoxy-1,5-dimethyl-4-hexenyl)-3-me-thoxy-1-chloromethyl-1-cyclohexanol; O-(3,5-dimethoxy-4-hydroxycinnamoyl)fumagillol or O-(chloracetyl-carbamoyl)fumagillol (TNP-470), and/or pharmaceutically acceptable salts thereof (e.g. O-(4-dimethylaminoethoxycinnamoyl)fumagillol oxalate).

Fumagillin, and some derivatives thereof, have a carboxylic acid moiety and can be administered in the form of the free acid. Alternatively, contemplated herein are pharmaceutically acceptable salts of fumagillin, fumagillol, and derivatives thereof.

Pharmaceutically acceptable salts illustratively include those that can be made using the following bases: ammonia, L-arginine, benethamine, benzathene, betaine, bismuth, calcium hydroxide, choline, deanol, diethanolamine, diethylamine, 2-(diethylamino)ethanol, ethylenediamine, N-methylglucamine, hydrabamine, 1H-imidazole, lysine, magnesium hydroxide, 4-(2-hydroxyethyl)morpholine, piperazine, potassium hydroxide, 1-(2-hydroxyethyl)pyrrolidine, sodium hydroxide, triethanolamine, zinc hydroxide, dicyclohexlamine, or any other electron pair donor (as described in Handbook of Pharmaceutical Salts, Stan & Wermuth, VHCA and Wiley, Uchsenfurt-Hohestadt Germany, 2002). Contemplated pharmaceutically acceptable salts may include hydrochloric acid, bromic acid, sulfuric acid, phosphoric acid, nitric acid, formic acid, acetic acid, trifluoroacetic acid, oxalic acid, fumaric acid, tartaric acid, maleic acid, methanesulfonic acid, benzenesulfonic acid or para-toluenesulfonic acid.

Esters of the present invention may be prepared by reacting fumagillin or fumagillol with the appropriate acid under standard esterification conditions described in the literature (Houben-Weyl 4th Ed. 1952, Methods of Organic Synthesis). Suitable fumagillin esters include ethyl methanoate, ethyl ethanoate, ethyl propanoate, propyl methanoate, propyl ethanoate, and methyl butanoate.

In another embodiment, contemplated irreversible inhibitors of MetAP2 may include a siRNA, shRNA, an antibody or an antisense compound of MetAP2.

Further examples of reversible and irreversible MetAP2 inhibitors are provided in the following references, each of which is hereby incorporated by reference: Olson et al. (U.S. Pat. No. 7,084,108 and WO 2002/042295), Olson et al. (U.S. Pat. No. 6,548,477; U.S. Pat. No. 7,037,890; U.S. Pat. No. 7,084,108; U.S. Pat. No. 7,268,111; and WO 2002/042295), Olson et al. (WO 2005/066197), Hong et al. (U.S. Pat. No. 6,040,337), Hong et al. (U.S. Pat. No. 6,063,812 and WO 1999/059986), Lee et al. (WO 2006/080591), Kishimoto et al. (U.S. Pat. No. 5,166,172; U.S. Pat. No. 5,698,586; U.S. Pat. Nos. 5,164,410; and 5,180,738), Kishimoto et al. (U.S. Pat. No. 5,180,735), Kishimoto et al. (U.S. Pat. No. 5,288,722), Kishimoto et al. (U.S. Pat. No. 5,204,345), Kishimoto et al. (U.S. Pat. No. 5,422,363), Liu et al. (U.S. Pat. No. 6,207,704; U.S. Pat. No. 6,566,541; and WO 1998/056372), Craig et al. (WO 1999/057097), Craig et al. (U.S. Pat. No. 6,242,494), BaMaung et al. (U.S. Pat. No. 7,030,262), Comess et al. (WO 2004/033419), Comess et al. (US 2004/0157836), Comess et al. (US 2004/0167128), Henkin et al. (WO 2002/083065), Craig et al. (U.S. Pat. No. 6,887,863), Craig et al. (US 2002/0002152), Sheppard et al. (2004, Bioorganic & Medicinal Chemistry Letters 14:865-868), Wang et al. (2003, Cancer Research 63:7861-7869), Wang et al. (2007, Bioorganic & Medicinal Chemistry Letters 17:2817-2822), Kawai et al. (2006, Bioorganic & Medicinal Chemistry Letters 16:3574-3577), Henkin et al. (WO 2002/026782), Nan et al. (US 2005/0113420), Luo et al. (2003, J. Med. Chem., 46:2632-2640), Vedantham et al. (2008, J. Comb. Chem., 10:195-203), Wang et al. (2008, J. Med. Chem., 51:6110-20), Ma et al. (2007, BMC Structural Biology, 7:84) and Huang et al. (2007, J. Med. Chem., 50:5735-5742), Evdokimov et al. (2007, PROTEINS: Structure, Function, and Bioinformatics, 66:538-546), Garrabrant et al. (2004, Angiogenesis 7:91-96), Kim et al. (2004, Cancer Research, 64:2984-2987), Towbin et al. (2003, The Journal of Biological Chemistry, 278(52):52964-52971), Marino Jr. (U.S. Pat. No. 7,304,082), Kallender et al. (U.S. patent application number 2004/0192914), and Kallender et al. (U.S. patent application numbers 2003/0220371 and 2005/0004116).

For example, contemplated MetAP2 inhibitors may include:

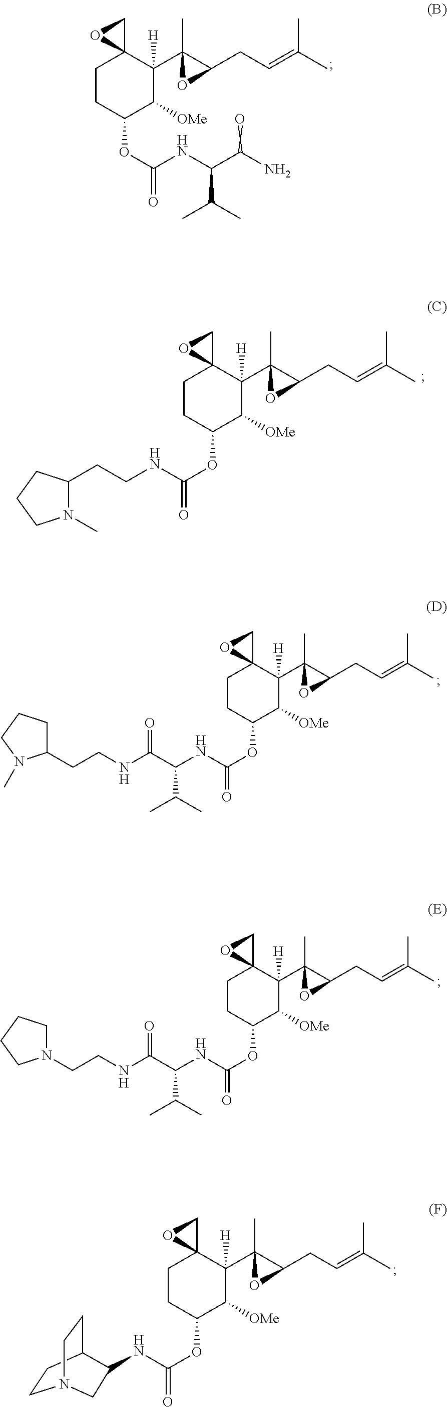

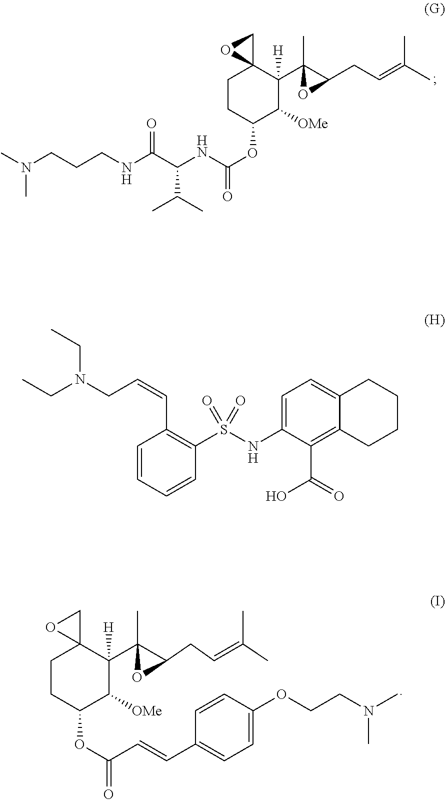

In some embodiments, a contemplated MetAP2 inhibitor may also modulate FGF21 (fibroblast growth factor 21). For example, a disclosed MetAP2 inhibitor e.g., fumagillin, may increase the plasma levels of FGF-21 in a subject after administration, e.g. after 3, 5, and/or 10 days or more of treatment.

In an embodiment, a contemplated MetAP2 inhibitor may modulate PCSK9 (proprotein convertase subtilisin/kexin type 9, an enzyme encoded by the PCSK9 gene. For example, contemplated MetAP2 inhibitors may reduce PSCK9 expression, which may increase LDL receptor levels on e.g., the surface of liver cells and may thus lead to increased LDL uptake and a lowering of plasma LDL-cholesterol.

### Methods

A method of treating, and/or mitigating or minimizing the risk of, cardiovascular and/or cerebrovascular disease in a subject in need thereof is provided herein, comprising parenterally or non-parenterally administering a therapeutically effective amount of a MetAP2 inhibitor to said subject. For example, contemplated methods include treatment of include atherosclerosis, heart attack, stroke, and/or heart failure, for example, hypertension, dyslipidemia, ischemic heart disease, cardiomyopathy, cardiac infarction, stroke, venous thromboembolic disease and/or pulmonary hypertension. For example, contemplated herein is a method of reducing serum levels of triglycerides and/or cholesterol in a patient in need thereof that includes administrating an effective amount of a MetAP2 inhibitor such as those disclosed herein. In some embodiments, such reduction of serum levels of triglycerides and/or cholesterol may improve risk of cardiovascular disease in a patient. In an embodiment, a method of increasing high density lipoproteins in a patient in need thereof is provided. Such patients or subjects subject to treatment may also be suffering from diabetes (e.g. type 2) and/or obesity. In some embodiments, a contemplated therapeutically effective amount of a MetAP2 as described below, does not substantially modulate or suppress angiogenesis, but is still effective as a MetAP2 inhibitor. The term “angiogenesis” is known to persons skilled in the art, and refers to the process of new blood vessel formation, and is essential for the exponential growth of solid tumors and tumor metastasis.

Also provided herein treating are methods of treating diabetic (e.g. type 2 diabetic) patients (or e.g. a patient suffering from a condition wherein administration of niacin is contradicted) also suffering from hypercholesterolemia, hyperlipidemia and/or hypoalphalipoproteinemia, comprising administering a MetAP2 inhibitor, and optionally, niacin and/or another GPR109A modulator or agonist. In some embodiments, such administration of MetAP2 inhibitors may minimize unwanted side effects from administration of e.g. niacin, e.g. flushing and/or impairment of glucose tolerance. In another embodiment, provided herein are methods of treating hyperlipidemia and/or hypercholesterolemia in a patient comprising administering a MetAP2 inhibitor, and one or more other therapeutic agents such as niacin, a statin (e.g. simvastatin, atorvastatin, cerivastatin, fluvastatin, lovastatin, mevastatin, pitavastatin, and/or rosuvastatin), a fibrate such as bezafibrate, ciprofibrate, clofibrate, gemfibrozil, and/or fenofibrate, an ACE inhibitor, and/or cholesterol absorption inhibitor (e.g. ezetimibe).

Other contemplated therapeutic agents that may be administered may include but are not limited to alpha blockers, e.g., terazosin (Hytrin®), doxazosin (Cardura®), tamsulosin (Flomax®) and alfuzosin (Uroxatral®), 5-alpha reductase inhibitors, e.g., finasteride (Proscar®) and dutasteride (Avodart®), saw palmetto, beta-sitosterol, and pygeum.

In some embodiments, co-administration of a MetAP-2 inhibitor and another active agent (e.g. niacin) occur at the same time. In other embodiments, administration of a MetAP-2 inhibitor occurs immediately prior to or immediately administration of another active agent. In yet another embodiment, a period of time may elapse between administration of a MetAP-2 inhibitor and another agent.

Contemplated other agents include those administered to treat type 2 diabetes such as sulfonylureas (e.g., chlorpropamide, glipizide, glyburide, glimepiride); meglitinides (e.g., repaglinide and nateglinide); biguanides (e.g., metformin); thiazolidinediones (rosiglitazone, troglitazone, and pioglitazone); glucagon-like 1 peptide mimetics (e.g. exenatide and liraglutide); sodium-glucose cotransporter inhibitors (e.g., dapagliflozin), renin inhibitors, and alpha-glucosidase inhibitors (e.g., acarbose and meglitol), dipeptidyl peptidase 4-inhibitors (e.g. sitagliptin. Vildagliptin, and saxagliptin), agonists of the GPR119 receptor and/or those administered to treat cardiac disorders and conditions, for example, chlorthalidone; hydrochlorothiazide; indapamide, metolazone; loop diuretics (e.g., bumetanide, ethacrynic acid, furosemide, lasix, torsemide); potassium-sparing agents (e.g., amiloride hydrochloride, spironolactone, and triamterene); peripheral agents (e.g., reserpine); central alpha-agonists (e.g., clonidine hydrochloride, guanabenz acetate, guanfacine hydrochloride, and methyldopa); alpha-blockers (e.g., doxazosin mesylate, prazosin hydrochloride, and terazosin hydrochloride); beta-blockers (e.g., acebutolol, atenolol, betaxolol, nisoprolol fumarate, carteolol hydrochloride, metoprolol tartrate, metoprolol succinate, Nadolol, penbutolol sulfate, pindolol, propranolol hydrochloride, and timolol maleate); combined alpha- and beta-blockers (e.g., carvedilol and labetalol hydrochloride); direct vasodilators (e.g., hydralazine hydrochloride and minoxidil); calcium antagonists (e.g., diltiazem hydrochloride and verapamil hydrochloride); dihydropyridines (e.g., amlodipine besylate, felodipine, isradipine, nicardipine, nifedipine, and nisoldipine); other ACE inhibitors (benazepril hydrochloride, captopril, enalapril maleate, fosinopril sodium, lisinopril, moexipril, quinapril hydrochloride, ramipril, trandolapril); angiotensin II receptor blockers (e.g., losartan potassium, valsartan, and Irbesartan); and combinations thereof.

### Administration and Formulation

Contemplated herein are formulations suitable for parenteral or non-parenteral administration of MetAP2 inhibitors. In certain embodiments, a subject may have a lower systemic exposure (e.g. at least about 2, 3, 5, 10, 20, or at least about 30% less systemic exposure) to the non-parenterally (e.g. orally) administered of a MetAP2 inhibitor as compared to a subject parenterally (e.g. subcutaneously) administered the same dose of the MetAP2 inhibitor.

Contemplated non-parenteral administration includes oral, buccal, transdermal (e.g. by a dermal patch), topical, inhalation, sublingual, ocular, pulmonary, nasal, or rectal administration.

Contemplated parenteral administration includes intravenous and subcutaneous administration, as well as administration at a site of a minimally-invasive procedure or a surgery.

In another embodiment, provided herein are effective dosages, e.g. a daily dosage of a MetAP2 inhibitor, that may not substantially modulate or suppress angiogenesis. For example, provided here are methods that include administering doses of MetAP2 inhibitors that are effective for e.g. reducing lipids or cholesterol, but are significantly smaller doses than that necessary to modulate and/or suppress angiogenesis (which may typically require about 12.5 mg/kg to about 50 mg/kg or more). For example, contemplated dosage of a MetAP2 inhibitor in the methods described herein may include administering about 25 mg/day, about 10 mg/day, about 5 mg/day, about 3 mg/day, about 2 mg/day, about 1 mg/day, about 0.75 mg/day, about 0.5 mg/day, about 0.1 mg/day, about 0.05 mg/day, or about 0.01 mg/day.

For example, an effective amount of the drug for reducing cholesterol or lipids in a patient in need thereof may be about 0.0001 mg/kg to about 25 mg/kg of body weight per day. For example, a contemplated dosage may from about 0.001 to 10 mg/kg of body weight per day, about 0.001 mg/kg to 1 mg/kg of body weight per day, about 0.001 mg/kg to 0.1 mg/kg of body weight per day or about 0.005 to about 0.04 mg/kg or about 0.005 to about 0.049 mg/kg of body weight a day. In an embodiment a MetAP2 inhibitor such as disclosed herein (e.g. O-(4-dimethlyaminoethoxycinnamoyl)fumagillol), may be administered about 0.005 to about 1 mg/kg, or to about 5 mg/kg, or about 0.005 to about 0.1 mg/kg of a subject.

For example, provided herein is a method for treating or reducing the risk of a cardiovascular disease in a subject in need thereof, comprising administering, parenterally (e.g. intravenously) or non-parenterally, about 0.005 to about 1 mg/kg, or about 0.005 to about 1.0 mg/kg or to 0.005 to about 0.05 mg/kg of a MetAP2 inhibitor, selected from O-(4-dimethylaminoethoxycinnamoyl)fumagillol and pharmaceutically acceptable salts thereof (for example, an oxalate salt), to said subject.

Contemplated methods may include administration of a composition comprising a MetAP2 inhibitor, for example, hourly, twice hourly, every three to four hours, daily, twice daily, 1, 2, 3 or 4 times a week, every three to four days, every week, or once every two weeks depending on half-life and clearance rate of the particular composition or inhibitor.

Treatment can be continued for as long or as short a period as desired. The compositions may be administered on a regimen of, for example, one to four or more times per day. A suitable treatment period may be, for example, at least about one week, at least about two weeks, at least about one month, at least about six months, at least about 1 year, or indefinitely. A treatment regimen may include a corrective phase, during which a MetAP2 inhibitor dose sufficient to provide e.g., reduction of lipids is administered, followed by a maintenance phase, during which a lower MetAP2 inhibitor dose sufficient to reduce or prevent increase in lipid and/or cholesterol levels is administered.

For pulmonary (e.g., intrabronchial) administration, MetAP2 inhibitors may be formulated with conventional excipients to prepare an inhalable composition in the form of a fine powder or atomizable liquid. For ocular administration, MetAP2 inhibitors may be formulated with conventional excipients, for example, in the form of eye drops or an ocular implant. Among excipients useful in eye drops are viscosifying or gelling agents, to minimize loss by lacrimation through improved retention in the eye.

Liquid dosage forms for oral or other administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the ocular, oral, or other systemically-delivered compositions can also include adjuvants such as wetting agents, and emulsifying and suspending agents.

Dosage forms for topical or transdermal administration of an inventive pharmaceutical composition may include ointments, pastes, creams, lotions, gels, powders, solutions, sprays, inhalants, or patches. The active agent is admixed under sterile conditions with a pharmaceutically acceptable carrier and any needed preservatives or buffers as may be required. For example, cutaneous routes of administration are achieved with aqueous drops, a mist, an emulsion, or a cream.

Transdermal patches may have the added advantage of providing controlled delivery of the active ingredients to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

When administered in lower doses, injectable preparations are also contemplated herein, for example, sterile injectable aqueous or oleaginous suspensions may be formulated according to the known art using suitable dispersing or wetting agents and suspending agents.

Compositions for rectal administration may be suppositories which can be prepared by mixing a MetAP2 inhibitor with suitable non-irritating excipients or carriers such as cocoa butter, polyethylene glycol or a suppository wax which are solid at ambient temperature but liquid at body temperature and therefore melt in the rectum and release the active agent(s). Alternatively, contemplated formulations can be administered by release from a lumen of an endoscope after the endoscope has been inserted into a rectum of a subject.

Oral dosage forms, such as capsules, tablets, pills, powders, and granules, may be prepared using any suitable process known to the art. For example, a MetAP2 inhibitor may be mixed with enteric materials and compressed into tablets.

Alternatively, formulations of the invention are incorporated into chewable tablets, crushable tablets, tablets that dissolve rapidly within the mouth, or mouth wash.

### EXAMPLES

This example is not intended in any way to limit the scope of this invention but is provided to illustrate aspects of the disclosed methods. Many other embodiments of this invention will be apparent to one skilled in the art.

### Example 1

### Administration of Fumagillin Increases Concentration of Betahydroxybutryrate in Mammals

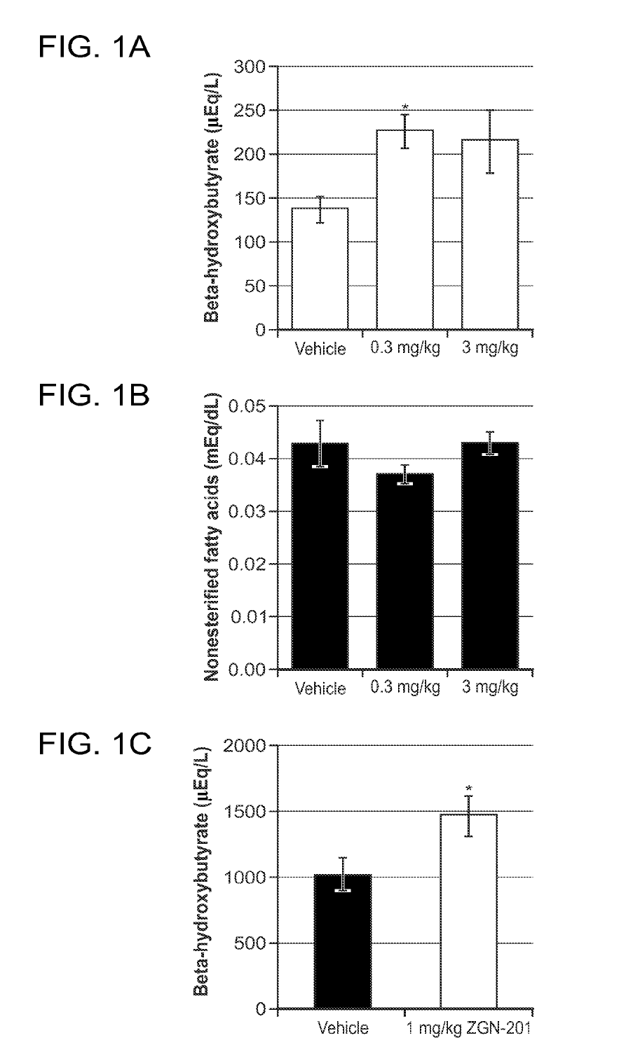

Oral administration of the MetAP2 inhibitor fumagillin causes an increase in circulating ketone bodies, as measured by concentration of beta hydroxybutyrate, which indicates an increase in the breakdown of stored fat and activation of ketogenesis. C57BL/6 mice conditioned on a high fat diet (also referred to as a diet-induced obese diet or DIO) were treated with fumagillin (ZGN-201) administered by oral gavage at doses of 0.3 or 3 mg/kg daily for 10 days resulting in an increase in beta hydroxybutyrate (FIG. 1A) concentrations, without an increase in free fatty acid concentration (FIG. 1B) as well as a reduction in body weight by 7 to 15 percent (data not shown). Increased hydroxybutyrate concentration persisted at longer time points, as was seen in C57BL/6 mice conditioned on a high fat diet and treated with fumagillin (ZGN-201) as a diet admixture at a dose of 1 mg/kg daily for 250 days (FIG. 1C).

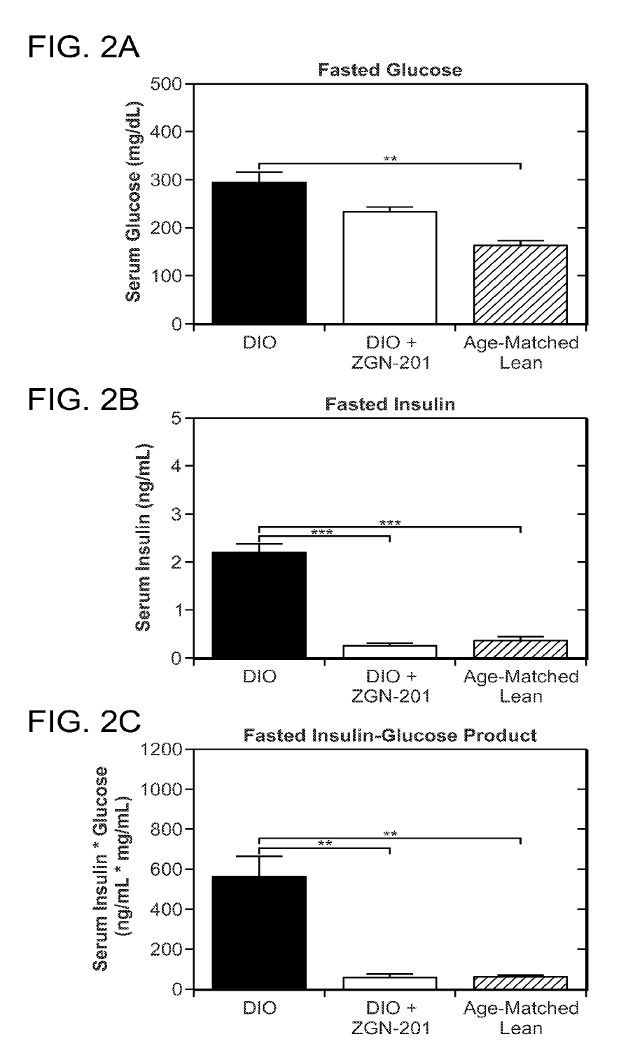

Fasted glucose and insulin levels, as well as insulin-glucose product, in fumagillin-treated mice was more similar to that of age-matched lean mice than that of untreated mice on a high fat diet (FIG. 2), indicating that fumagillin (ZGN-201) normalizes sensitivity to insulin in mice on a high fat diet.

### Example 2

### Plasma LDL Cholesterol in Humans with Elevated Total Cholesterol Levels

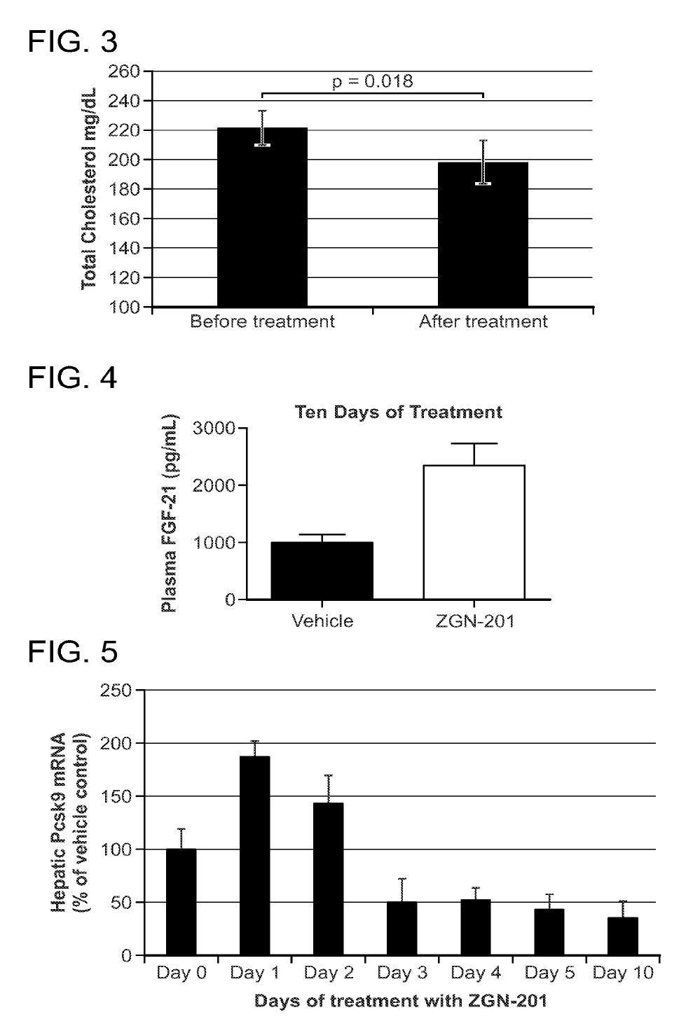

Human subjects with initial fasting total cholesterol levels in excess of 180 mg/dl were administered Compound A twice weekly for a total of four weeks of treatment. Total cholesterol levels were reduced by treatment (n=10 subjects, p=0.018 by paired Student's t test), as shown in FIG. 3.

### Example 3

Rats were administered 1 or 3 mg/kg fumagillol daily for a total of four weeks of treatment. Total cholesterol levels were reduced by treatment (n=10 rats, p<−0.004 by paired Williams' test):

| Treatment | n | Mean | SEM | % of vehicle | p |
|---|---|---|---|---|---|
| Vehicle (10% DMSO) | 10 | 86.7 | 3.8 | | |
| 3 ml/kg po | | | | | |
| Fumagillol (1 mg/kg po) | 10 | 66.3 | 3.7 | 76.5 | 0.004** |
| Fumagillol (3 mg/kg po) | 10 | 55.2 | 4.5 | 63.7 | <0.001*** |

### Example 4

### Analysis of FGF-21 in a Mouse Model

The following table shows results of a study using a R&D Systems Quantikine Mouse FGF-21 Immunoassay based on ten days of treatment with ZGN-201. In Table 1, animals #1.1, 1.2, 1.3, 2.1, 2.2 and 2.3 were vehicle-treated and animal #3.1, 3.2, 3.3, 4.1, 4.2 and 4.3 were ZGN-201-treated. Sample 3.1 was above the level of quantification.

TABLE 1

| | Abs 450 nm | Concentrations | Abs 450 nm | Concentrations | | | Average of | | |
|---|---|---|---|---|---|---|---|---|---|
| Animal # | 1:1 dilution | | 1:5 dilution | | Absolute numbers in pg/ml | | duplicates | MEAN | SEM |
| 1.1 | 0.751 | 726.10 | 0.31 | 266.55 | 1452.20 | 1332.77 | 1392.48 | | |
| 1.2 | 0.465 | 426.71 | 0.22 | 174.43 | 853.43 | 872.17 | 862.80 | | |
| 1.3 | 0.293 | 246.66 | 0.16 | 102.21 | 493.33 | 511.03 | 502.18 | | |
| 2.1 | 0.444 | 404.73 | 0.29 | 240.38 | 809.46 | 1201.92 | 1005.69 | | |
| 2.2 | 0.768 | 743.90 | 0.31 | 264.46 | 1487.79 | 1322.30 | 1405.05 | | |
| 2.3 | 0.474 | 436.14 | 0.19 | 137.80 | 872.27 | 688.98 | 780.63 | 991.47 | 145.20 |
| 3.1 | 3.433 | >2000 | 2.31 | >2000 | AQL | AQL | AQL | | |
| 3.2 | 1.191 | 1186.69 | 0.46 | 424.62 | 2373.39 | 2123.10 | 2248.25 | | |
| 3.3 | 1.218 | 1214.96 | 0.55 | 519.88 | 2429.92 | 2599.40 | 2514.66 | | |
| 4.1 | 1.038 | 1026.53 | 0.49 | 456.03 | 2053.07 | 2280.13 | 2166.60 | | |
| 4.2 | 0.617 | 585.83 | 0.29 | 243.52 | 1171.66 | 1217.62 | 1194.64 | | |
| 4.3 | 1.63 | 1646.24 | 0.80 | 780.53 | 3292.48 | 3902.67 | 3597.58 | 2344.34 | 384.85 |

FIG. 4 indicates the average (±SEM) plasma FGF-21 levels for all study groups.

### Example 5

### Reduction of PCSK9 Expression

Liver mRNA levels of PCSK9 were examined in C57BL/6 mice treated with ZGN-201. FIG. 5 depicts the hepatic PCSK9 mRNA (% of vehicle control) vs. days of treatment with ZGN-201. FIG. 5 indicates that at day 3 and after, administration of ZGN-201 leads to reduced PCSK9 expression.

### Example 6

### Reduction of C-Reactive Protein (CRP) Levels

Obese patients were treated in three cohorts with intravenous administration of a formulation of the compound 6-O-(4-dimethylaminoethoxy)cinnamoyl fumagillol oxalate. The compound was intravenously administered to each patient of a cohort (except for a placebo cohort) twice weekly for 26 days. Each of patients in the three non-placebo cohorts received either 0.1 mg/m² (cohort 1); 0.3 mg/m² (cohort 2); or 0.9 mg/m² (cohort 3) doses of the compound at the time of administration. The trial was conducted under the appropriate government and medical supervision.

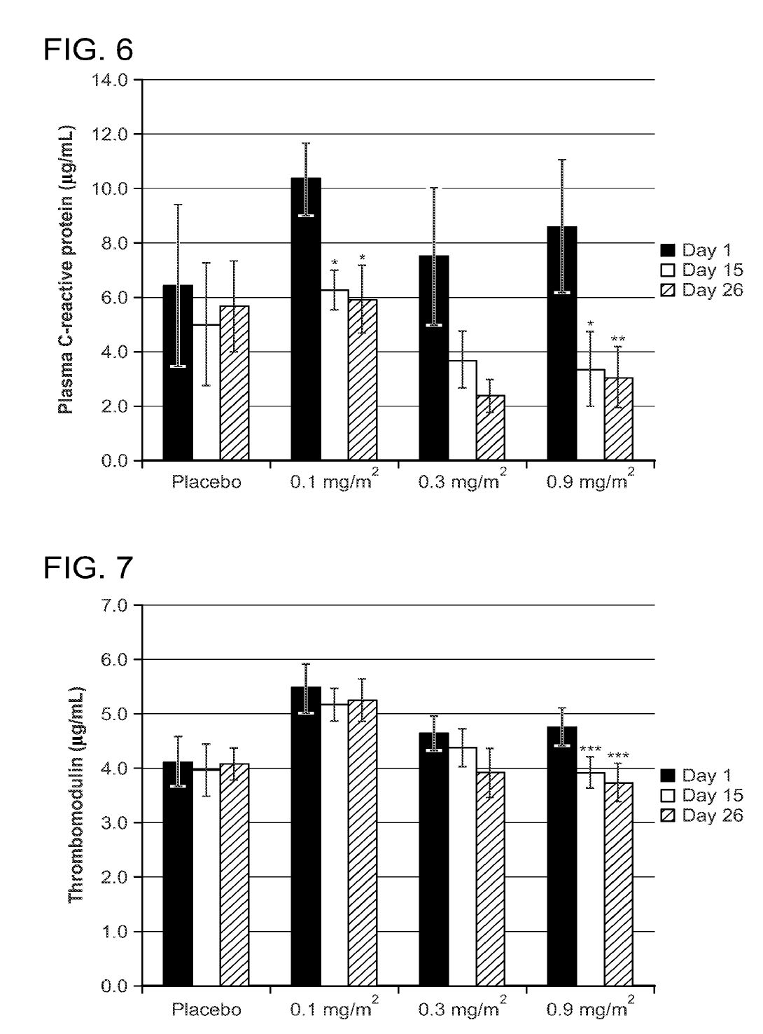

As shown in FIG. 6, administration of 6-O-(4-dimethylaminoethoxy)cinnamoyl fumagillol oxalate causes a decrease in levels of C-Reactive Protein (CRP), a key inflammatory marker used in humans as an indicator of risk of cardiovascular disease, in patients' serum. Notably, treatment with the compound at the 0.9 mg/m² dose level resulted in six out of eight subjects with elevated CRP concentrations (above 3.0 μg/ml) being reduced to levels within the normal range by the 26^{th} day of treatment (p<0.01 by paired Student's t test). This result indicates that administration of a MetAP2 inhibitor decreases risk of cardiovascular disease. Surprisingly, this reduction was independent of effects on metabolic parameters and body weight; i.e., there was no real relationship between weight loss and decrease in CRP.

### Example 7

### Reduction of Thrombomodulin

The expression of another cardiovascular risk marker, thrombomodulin, was examined in the patients described in Example 6. FIG. 7 shows that administration of 6-O-(4-dimethylaminoethoxy)cinnamoyl fumagillol oxalate decreases thrombomodulin levels, indicating that administration of a MetAP2 inhibitor decreases risk of cardiovascular disease. Similarly to Example 6, the reduction in thrombomodulin was independent of effects on metabolic parameters and body weight.

### Example 8

### Reduction of Low Density Lipoprotein (LDL) Cholesterol

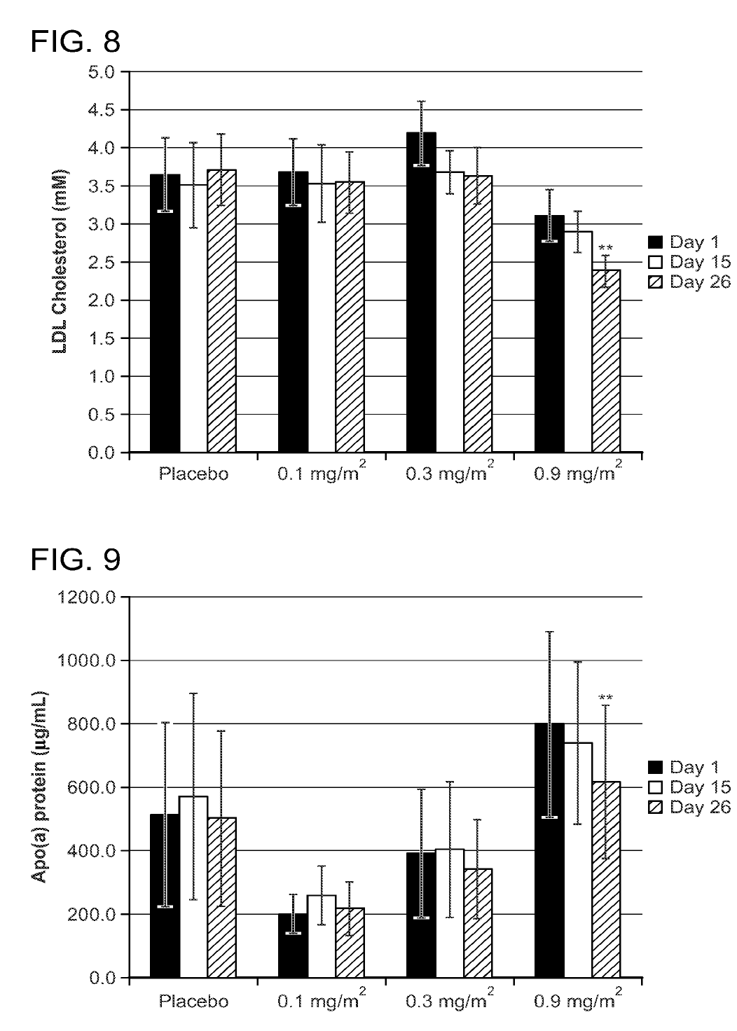

The expression of another cardiovascular risk marker, low density lipoprotein cholesterol (LDLc), was examined in the patients described in Example 6. FIG. 8 shows that administration of 6-O-(4-dimethylaminoethoxy)cinnamoyl fumagillol oxalate decreases LDL-c levels, indicating that administration of a MetAP2 inhibitor decreases risk of cardiovascular disease. Similarly to Example 6, the reduction in LDL-c was independent of effects on body weight.

### Example 9

### Reduction of Apolipoprotein(a) (apoLp(a))

The expression of another cardiovascular risk marker, apolipoprotein(a) (apoLp(a)), was examined in the patients described in Example 6. FIG. 9 shows that administration of 6-O-(4-dimethylaminoethoxy)cinnamoyl fumagillol oxalate decreases apoLp(a) levels, indicating that administration of a MetAP2 inhibitor decreases risk of cardiovascular disease. Similarly to Example 6, the reduction in apoLp(a) was independent of effects on body weight.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method of treating, or minimizing the risk of, cardiovascular or cerebrovascular disease in a patient in need thereof, comprising administering to said patient an therapeutically effective amount of a MetAP2 inhibitor thereby increasing serum levels of high density lipoproteins in said patient, wherein said therapeutically effective amount does not substantially modulate or suppress angiogenesis.

2. The method of claim 1, wherein the cardiovascular disease or cerebrovascular disease is selected from the group consisting of atherosclerosis, heart attack, stroke, or heart failure.

3. The method of claim 1, wherein the patient is obese.

4. The method of claim 1, wherein the patient is suffering from diabetes.

5. A method of reducing triglycerides in the serum of a patient in need thereof, comprising administering to said patient a therapeutically effective amount of a MetAP2 inhibitor, wherein said therapeutically effective amount does not substantially modulate or suppress angiogenesis.

6. The method of claim 5, wherein the patient is obese.

7. The method of claim 6, wherein the patient is suffering from diabetes.

8. The method of claim 5, wherein administration of niacin or a GPR109a receptor agonist is contradicted in said patient.

9. The method of claim 1, wherein said method minimizes side effects or risks associated with blood vessel growth or maturation.

10.-20. (canceled)

21. The method of claim 1, wherein said MetAP2 inhibitor is selected from O-(4-dimethylaminoethoxycinnamoyl)fumagillol and pharmaceutically acceptable salts thereof.

22. The method of claim 1, wherein said MetAP2 inhibitor is a substantially reversible inhibitor.

23.-24. (canceled)

25. A method of treating hyperlipidemia and/or hypercholesterolemia in a patient in need thereof, comprising administering an effective amount of the following to said patient:
a) one or more therapeutic agents each selected from the group consisting of: niacin, a statin, a fibrate, an angiotension-converting enzyme inhibitor, and a cholesterol absorption inhibitor; and
b) a MetAP-2 inhibitor.

26. The method of claim 25, wherein the patient is suffering from type 1 or type 2 diabetes.

27. The method of claim 25, wherein said method minimizes flushing.

28. (canceled)

29. The method of claim 25, wherein the therapeutic agent is selected from the group consisting of ezetimibe, simvastatin, atorvastatin, cerivastatin, fluvastatin, lovastatin, mevastatin, pitavastatin, rosuvastatin, bezafibrate, ciprofibrate, clofibrate, gemfibrozil, and fenofibrate.

30. A method of treating hypercholesterolemia or hyperlipidemia in a patient in need thereof, comprising administering an effective amount of O-(4-dimethylaminoethoxycinnamoyl)fumagillol or pharmaceutically acceptable salts thereof.

31. The method of claim 30, wherein the serum level of high density lipoproteins is increased in said patient upon administration.

32. The method of claim 1, wherein the MetAP-2 inhibitor is a Compound I, represented by: 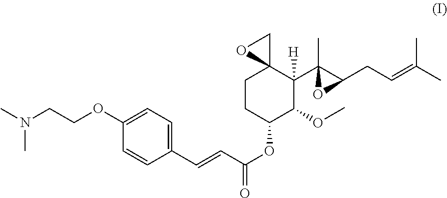 [SMILES: CO[C@@H]1[C@H](OC(=O)/C=C/c2ccc(OCCN(C)C)cc2)CC[C@]2(CO2)[C@H]1[C@@]1(C)O[C@@H]1CC=C(C)C]
or a pharmaceutically acceptable salt thereof.

33.-35. (canceled)
